(19) United States
(12) Patent Application Publication
(10) Pub. No.: US 2021/0040013 A1
(43) Pub. Date: Feb. 11, 2021

(54) MULTISTAGE ALKYLATION VIA BYPRODUCT REMOVAL

(71) Applicant: ExxonMobil Research and Engineering Company, Annandale, NJ (US)

(72) Inventors: Doron Levin, Highland Park, NJ (US); Vinit Choudhary, Cypress, TX (US); Matthew S. Mettler, Tomball, TX (US); Joshua W. Allen, Branchburg, NJ (US)

(21) Appl. No.: 16/928,850

(22) Filed: Jul. 14, 2020

Related U.S. Application Data
(60) Provisional application No. 62/883,260, filed on Aug. 6, 2019.

(51) Int. Cl. C07C 2/62; C07C 7/13; C07C 7/04; B01J 19/24

(52) U.S. Cl. CPC C07C 2/62; C07C 7/13; C07C 7/04; B01J 19/246; B01J 2219/2423; C07C 2529/04; B01J 2219/32203; B01J 2219/2419; B01J 19/2465

(57) ABSTRACT

The present disclosure is related to processes for the alkylation of an isoparaffin. The process may include introducing, in a multistage reactor, a solid acid catalyst including a zeolite to an isoparaffin feed and an olefin feed to form an alkylation product mixture including C5+ olefins. The processes may further include separating at least a portion of the C5+ olefins from the alkylation product mixture to form an oligomer light stream. The present disclosure further relates to multistage reactors for the alkylation of an isoparaffin with an olefin. The multistage reactors may include a plurality of stages, and a plurality of separation systems. The multistage reactors may also include an outlet space coupling each stage to a separation system and an inlet space coupling a separation system to a subsequent stage.

### CROSS-REFERENCE TO RELATED APPLICATIONS

This application claims the benefit of U.S. Provisional Application No. 62/883,260 filed Aug. 6, 2019, which is herein incorporated by reference in its entirety.

### FIELD OF THE INVENTION

The present disclosure relates to processes and apparatuses for alkylation of isoparaffins and, in particular, to processes and apparatuses for alkylation of isoparaffins with olefins to produce high octane rated additive for fuels, such as gasoline.

### BACKGROUND OF THE INVENTION

The alkylation of isoparaffins, such as isobutane, is an important refinery process for the production of high octane alkylate as a blend component for gasoline. Alkylation involves the addition of an alkyl group to an organic molecule. Thus, an isoparaffin can be reacted with an olefin to provide an isoparaffin of higher molecular weight. The product is a valuable blending component for gasoline due to its high octane rating, low sulfur, low olefin, and low aromatic content. Industrially, alkylation often involves the reaction of C2-C5 olefins with, for example, isobutane in the presence of an acidic catalyst to form alkylates. Alkylates are valuable blending components for the manufacture of premium gasolines due to their high octane ratings.

In the past, alkylation processes have included the use of liquid acids, such as hydrofluoric acid or sulfuric acid as catalysts. The use of liquid acids provides challenges in disposal of spent acid streams. Furthermore, consideration has been given by regulatory authorities to the restriction of the use of liquid acids in industrial alkylation reactions. An alternative to liquid acids are solid acids, such as zeolites. However, some solid acids, such as faujasite, typically have short catalyst lifetimes which lead to frequent catalyst regeneration and increased costs and may further require the use of precious metals such as platinum and palladium in catalyst regeneration.

Recent efforts in further improving alkylation catalysts over liquid acid catalysts and previous solid acid catalysts have been focused on the development and use of solid acid catalysts, including zeolites, such as zeolites having the MWW framework type, e.g. MCM-22, MCM-36 and MCM-49 for the catalytic alkylation of an olefin with an isoparaffin. (U.S. Pat. Nos. 4,992,615, 5,254,792, 5,304,698, 5,354,718, 5,516,962). The previous approaches in alkylation of isoparaffins focused on using a single stage reactor where the feed isobutane to olefin ratio (i:o ratio), a volume to volume ratio, was set by the composition of the gas entering the single stage reactor. For liquid acids the i:o ratio has typically been 4:1 to 10:1, and for solid catalysts the i:o ratio has typically been 40:1 to 50:1, both based solely on the composition of the feedstock entering a single stage reactor.

The use of a single stage reactor may limit the ability to convert olefins and isoparaffins into higher octane rated fuel additives. For example, a single stage reactor does not permit splitting of the olefin feedstock creating a lower local concentration of olefin and a greater i:o ratio.

There remains a need for improved isoparaffin-olefin alkylation processes that can be catalyzed by a solid acid catalyst with high conversion and high activity that maintains product quality of existing liquid phase processes.

### SUMMARY OF THE INVENTION

The present disclosure is related to processes for the alkylation of an isoparaffin. The process may include introducing, in a multistage reactor, a solid acid catalyst including a zeolite to an isoparaffin feed and an olefin feed to form an alkylation product mixture including C5+ olefins. The processes may further include separating at least a portion of the C5+ olefins from the alkylation product mixture to form an oligomer light stream.

The present disclosure further relates to multistage reactors for the alkylation of an isoparaffin with an olefin. The multistage reactors may include a first stage, a second stage, and a separation system. The multistage reactors may also include a first outlet space coupling the first stage and the separation system and a first inlet space coupling the separation system and the second stage. Additionally, the multistage reactors of the present disclosure may include a first inlet disposed upstream of the first stage and configured to receive an olefin feed, and a second inlet disposed upstream of the first stage and configured to receive an isoparaffin feed.

The present disclosure further relates to multistage reactors for the alkylation of an isoparaffin with an olefin. The multistage reactors may include a plurality of stages, and a plurality of separation systems. The multistage reactors may also include an outlet space coupling each stage to a separation system and an inlet space coupling a separation system to a subsequent stage.

### BRIEF DESCRIPTION OF THE DRAWING

FIG. 1A is a depiction of a reactor with one stage configured to receive an olefin feed and an isoparaffin feed.

FIG. 1B is a depiction of a reactor with two stages configured to receive an olefin feed and an isoparaffin feed, according to an embodiment.

FIG. 1C is a depiction of a reactor with four stages configured to receive an olefin feed and an isoparaffin feed, according to an embodiment.

FIG. 1D is a depiction of a reactor with eight stages configured to receive an olefin feed and an isoparaffin feed, according to an embodiment.

FIG. 2A is a depiction of a reactor with two stages and a separation system, according to one embodiment.

FIG. 2B is a depiction of a reactor with four stages and three separation systems, according to one embodiment.

### DETAILED DESCRIPTION OF THE INVENTION

Previous alkylation processes and systems relied on a single stage reactor. Single stage alkylation reactors may provide lower conversion of isoparaffins and olefins into higher octane rated fuel additives, increased by-product formation, and can be limited in flow rate or i:o ratio. It was believed that the addition of multiple stages would improve conversion because olefin interactions with active catalyst site could increase with additional stages including catalyst. Additionally, the use of multiple stages may allow for use of similar feeds, but provide a larger i:o ratio at each stage than a single stage reactor could provide. A multiple stage reactor may offer improvements over single stage processes including splitting of olefin introduction into various stages which decreases the local concentration of olefin in a catalyst bed, which may provide improved i:o ratios, decreased issues with catalyst deactivation, decreased byproduct formation, and improved conversion of isoparaffins. Improved conversion may result from increased olefin interactions with active catalyst sites resulting from passing over catalyst beds within additional reactor stages.

However, it has been discovered that performing the alkylation in multiple stages shows a decrease in conversion and increased loss of catalyst activity at each subsequent stage. Without being limited by theory, it is believed that olefin oligomerization creates higher olefins (C5+ olefins) that block catalyst active sites decreasing alkylation of paraffins and reducing catalyst activity. Furthermore, the feed entering a later stage does not have the same chemical composition as the feed entering previous stages because the feed after the first stage contains some quantity of higher olefins. Additionally, the problem of higher olefin content may be additive because a decrease in catalytic activity lowers isoparaffin conversion rates. At lower isoparaffin conversion rates more olefin oligomers are formed and, therefore, catalyst activity may decrease more quickly.

The benefits of a multistage reactor can be realized by removing the olefin oligomers produced via interstage distillation or adsorption.

It has been discovered that high conversion rates, maintenance of high catalyst activity, and decreased production of olefin oligomers can be achieved by byproduct removal via interstage distillation or adsorption after a stage of a multistage reactor. Additionally it has been discovered that a high boiling fraction (about 300° F. or greater) causes at least a portion of the catalyst deactivation. Because many olefin oligomers can be higher molecular weight than the desired products, the oligomers may be separated via distillation. Additionally or alternatively, the difference in molecular weight coupled with the remaining olefin bond of an oligomer may allow for separation via adsorption. Whether distillation or adsorption is employed, the removal of olefin oligomer byproducts in the interstage of a multistage reactor may decrease catalyst deactivation and increase conversion rates.

### Definitions

The term “Cn” compound (olefin or paraffin) where n is a positive integer, e.g., 1, 2, 3, 4, 5, etc., means a compound having n number of carbon atom(s) per molecule. The term “Cn+” compound where n is a positive integer, e.g., 1, 2, 3, 4, 5, etc., means a compound having at least n number of carbon atom(s) per molecule. The term “Cn−” compound where n is a positive integer, e.g., 1, 2, 3, 4, 5, etc., means a compound having no more than n number of carbon atom(s) per molecule.

The term “critical point” is the liquid-vapor end point of a phase equilibrium curve that designates conditions under which a liquid and vapor may coexist. At temperatures higher than the critical point (a “critical temperature”) a gas cannot be liquefied by pressure alone. At temperatures and pressures higher than the critical point the material is a supercritical fluid. For the purposes of this disclosure the critical point for isobutane is 134.6° C. and 3650 kPa, and the critical point for isopentane is 187.2° C. and 3378 kPa.

The term “molecular sieve” means a substance having pores of molecular dimensions that permit the passage of molecules below a certain size. Examples of molecular sieves include but are not limited to zeolites, silicoaluminophosphate molecular sieves, and the like.

### Reactor Design and Conditions

The processes described can be conducted in any suitable multistage reactor, such as one including fixed-beds, moving beds, swing beds, fluidized beds (including turbulent beds), and/or one or more combinations thereof. A reactor stage begins at the point in which olefin is introduced and ends at either an interstage space, where additional olefin is introduced, or where product mixture is removed. A multistage reactor may have one or more interstage spaces between stages. An interstage space may be an open space, a filled space, a separation barrier, a distribution plate or system, or an injection point. Multistage reactors of the present disclosure may be configured to receive an olefin feed at multiple sites or inlets, and the introduction of olefin marks a new reactor stage. Furthermore, multistage reactors may have one or more outlets for the removal of product mixtures. Each of these outlets may be coupled to one or more separation systems configured to remove higher olefins (C5+ olefins) from product mixtures. A separation system may include distillation apparatus(es) or an adsorption bed(s) to facilitate the removal of olefin oligomers. Distillation apparatuses or adsorption beds may be coupled to one or more reactor inlets or injection points.

In addition, the reactor may include multiple catalyst beds located in the same or different housing. A multistage reactor or a stage within the reactor may include a bed of catalyst particles where the particles have insignificant motion in relation to the bed (a fixed bed). In addition, injection of the olefin feed can be effected at a single point in the reactor or at multiple points spaced along the reactor. The isoparaffin feed and the olefin feed may be premixed before entering the reactor.

In certain embodiments of the present disclosure, the multistage reactor includes a plurality of fixed beds, continuous flow-type reactor stages in either a down flow or up flow mode, where the reactor stages may be arranged in series or parallel. The multistage reactor may include multiple reactor stages in series and/or in parallel, for example, a multistage reactor may include 2 stages, 4 stages, 8 stages, 10 stages, 12 stages, or another plurality of stages. A reactor stage includes a catalyst bed. The reactor stage may have various configurations such as: multiple horizontal beds, multiple parallel packed tubes, multiple beds each in its own reactor shell, or multiple beds within a single reactor shell. In certain embodiments, a reactor stage includes a fixed bed which provides uniform flow distribution over the entire width and length of the bed to utilize substantially all of the catalyst. In at least one embodiment, the multistage reactor can provide heat transfer from reactor stages or catalyst beds in order to provide effective methods for controlling temperature.

The efficiency of a multistage reactor containing fixed beds of catalyst may be affected by the pressure drop across a fixed bed. The pressure drop depends on various factors such as the path length, the catalyst particle size, and pore size. A pressure drop that is too large may cause channeling through the catalyst bed, poor efficiency. In some embodiments, the reactor has a cylindrical geometry with axial flows through the catalyst beds.

The various designs of the multistage reactor may accommodate control of specific process conditions, e.g. pressure, temperature, LHSV, and OLHSV (olefin liquid hourly space velocity). The combination of LHSV and OLHSV determine catalyst volume and residence time that may provide the desired conversion.

Operating pressures may be controlled to reduce or eliminate oligomerization reactions and/or favor alkylation reactions. Additionally, increased reactor pressures may improve conversion rates for the olefin feed and improve selectivity towards the alkylated paraffin over olefin oligomers. Operating pressure may be from about 300 to about 1500 psig (about 2068 to about 10342 kPag), such as from about 400 to about 1200 psig (about 2758 to about 8274 kPag), from about 450 psig to about 1000 psig (about 3102 to about 6895 kPag), from about 550 psig to about 950 psig (about 3792 to about 6550 kPag), from about 650 psig to about 950 psig (about 4482 to about 6550 kPag), from about 750 psig to about 950 psig (about 5171 to about 6550 kPag), or from about 800 psig to about 950 psig (about 5515 to about 6550 kPag). In some embodiments, the operating temperature and pressure remain above the critical point for the isoparaffin feed during the reactor run.

Additionally, operating temperatures may be controlled to reduce or eliminate olefin oligomerization reactions and/or favor alkylation of isoparaffins. Operating temperature may be from about 100° C. or greater, such as about 130° C. or greater, about 140° C. or greater, about 150° C. or greater, or about 160° C. or greater, such as from about 100° C. to about 200° C., from about 130° C. to about 170° C., or from about 140° C. to about 160° C. Operating temperatures may exceed the critical temperature of the isoparaffin feed, or the principal component in the isoparaffin feed. The term “principal component” is defined as the component of highest concentration in the feedstock. For example, isobutane is the principal component in a feedstock consisting of isobutane and 2-methylbutane in isobutane:2-methylbutane weight ratio of about 50:1.

The temperature of the multistage reactor or an individual stage within the reactor may affect by-product formation and a temperature higher than 130° C. may decrease heavier olefin concentrations. Furthermore, an increase in temperature may improve conversion of the olefin feed. However, for certain olefins, a higher temperature increases olefin isomerization, and olefin isomerization may lead to the formation of alkylation products that are lower in value. For example, in the alkylation of isobutane with 2-butene, a main component of the alkylation product mixture is trimethylpentane which has an octane rating of 100, but if 2-butene is isomerized to 1-butene the alkylation shifts to higher production of dimethylhexane which has an octane rating of 70, providing less value as a fuel additive. Therefore, temperature may be used to reduce or eliminate heavier olefin concentrations, especially in cases where the olefin is not affected by isomerization, such as propene or isobutene. In some embodiments, the alkylation product mixture contains about 10 wt % or less, such as about 5 wt % or less, about 2 wt % or less, about 1 wt % or less, or is substantially free of products of olefin oligomerization.

Hydrocarbon flow through a reactor stage containing the catalyst is typically controlled to provide an olefin liquid hourly space velocity (OLHSV) sufficient to convert about 99 percent, or more, by weight of the fresh olefin to alkylation product. In some embodiments, OLHSV values are from about 0.01 hr⁻¹ to about 10 hr⁻¹, such as about 0.02 hr⁻¹ to about 1 hr⁻¹, or such as about 0.03 hr⁻¹ to about 0.1 hr⁻¹. The liquid hourly space velocity of the isoparaffin is controlled to meet a target i:o ratio. Because the i:o ratio is vol:vol, the isoparaffin liquid hourly space velocity is directly correlated to the OLHSV.

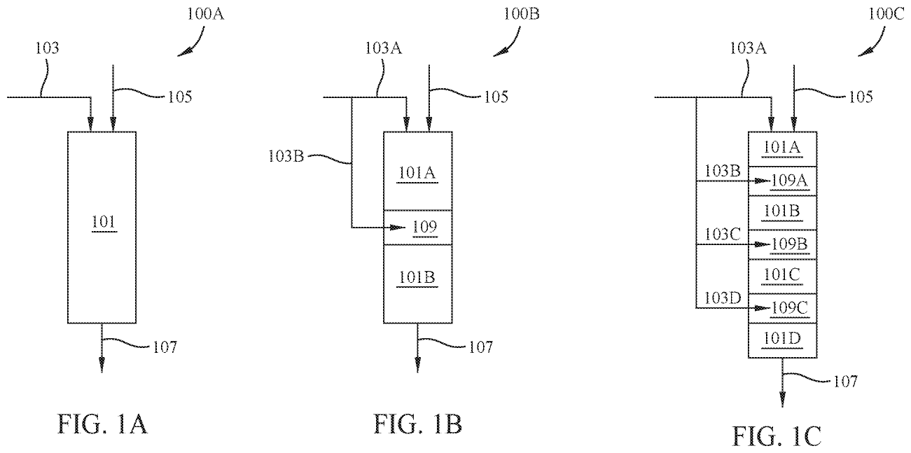

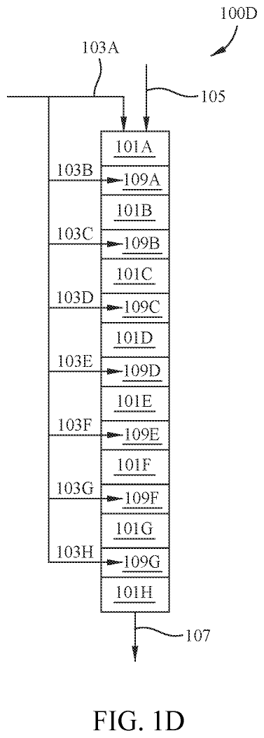

FIG. 1A depicts an alkylation reactor 100A with a single reactor stage 101. Reactor stages(s) may individually or collectively be termed an alkylation zone and include catalyst, such as a solid acid catalyst including one or more zeolites, which may further include crystalline microporous materials of the MWW framework type. The olefin feed is introduced to reactor stage 101 via line 103 and the isoparaffin feed through line 105. An alkylation product mixture exits the reactor through line 107. In alkylation reactor 100A the i:o ratio is controlled solely by the composition of the olefin feed and the isoparaffin feed entering reactor bed 101.

FIG. 1B depicts a multistage alkylation reactor 100B with two reactor stages: first stage 101A and second stage 101B. The olefin feed is introduced to the reactor beds via lines 103A and 103B and OLHSV values are from about 0.01 hr⁻¹ to about 10 hr⁻¹, such as about 0.02 hr⁻¹ to about 1 hr⁻¹, or such as about 0.03 hr⁻¹ to about 0.1 hr⁻¹. The split introduction of the olefin feed allows a lower concentration (half) of the olefin feed to be introduced locally to each of the first stage 101A and the second stage 101B. The isoparaffin feed is introduced to alkylation reactor 100B through line 105. Alkylation reactor 100B has an interstage space 109 between first stage 101A and the second stage 101B to allow for introduction of additional olefin feed through line 103B. If lines 103 and 105 have the same composition as in FIG. 1A, then the local i:o ratio is doubled in the configuration of FIG. 1B because the olefin feed is divided into two lines 103A and 103B and the olefin introduced via line 103A to first stage 101A can be converted, such as about 90 wt % or greater, 95 wt % or greater, 98 wt % or greater, or 99 wt % or greater is converted in the reaction within first stage 101A, based on the total weight of olefin in the olefin feed introduced via line 103A. Additionally, only a small portion of the isoparaffin feed is converted by the reaction in first stage 101A, such as about 10 wt % or less, 5 wt % or less, 2 wt % or less, 1 wt % or less, 0.5 wt % or less, or 0.1 wt % or less of the isoparaffin feed is converted based on the total weight of isoparaffin. Therefore, the amount of isoparaffin introduced to interstage 109 and, therefore, introduced to second stage 101B is similar to that introduced to first stage 101A. For example, if an i:o ratio of 100:1 is introduced to first stage 101A and there is an olefin conversion of 100% then the i:o ratio in second stage 101B would be ˜99:1, if no additional isoparaffin was added. Furthermore, the olefin introduced to interstage 109 (either via line 103B or from the effluent of first stage 101A) and, therefore, introduced to second stage 101B is similar in quantity to that introduced to first stage 101A. Additionally, because the selected isoparaffin may be consumed in each stage, such as in amounts of about 10 wt % or less, about 5 wt % or less, about 2 wt % or less, about 1 wt % or less, about 0.5 wt % or less, or about 0.1 wt % or less, additional isoparaffin may be added in an interstage space so as to maintain a consistent i:o ratio throughout the multistage reactor. Similarly to FIG. 1A, an alkylation product mixture exits the reactor through line 107.

FIG. 1C depicts a multistage alkylation reactor 100C with four reactor stages: first stage 101A, second stage 101B, third stage 101C, and fourth stage 101D. The olefin feed is introduced to the reactor beds via lines 103A, 103B, 103C and 103D and OLHSV values are from about 0.01 hr⁻¹ to about 10 hr⁻¹, such as about 0.02 hr⁻¹ to about 1 hr⁻¹, or such as about 0.03 hr⁻¹ to about 0.1 hr⁻¹. The split introduction of the olefin feed allows a lower concentration (one quarter) of the olefin feed to be introduced locally to each of the first stage 101A, second stage 101B, third stage 101C, and fourth stage 101D. The isoparaffin feed is introduced to alkylation reactor 100C through line 105. Alkylation reactor 100C has multiple interstage spaces: first interstage space 109A, second interstage space 109B, and third interstage space 109C between reactor stages 101A, 101B, 101C, and 101D to allow for introduction of additional olefin feed through lines 103B, 103C, and 103D. If lines 103 and 105 have the same composition as in FIG. 1A, then the local i:o ratio is 4 times that found in FIG. 1A, because the olefin feed is divided into four lines 103A, 103B, 103C, and 103D. The i:o ratio in a single stage is only slightly affected by prior stage(s) because the olefin introduced to a prior stage can be largely converted within that stage, but the isoparaffin is introduced at such a ratio that the amount converted may have little effect on the ratio in later stages. For example, the olefin introduced via line 103A to first stage 101A is converted, such as about 90 wt % or greater, 95 wt % or greater, 98 wt % or greater, or 99 wt % or greater is converted in first stage 101A, based on the total weight of olefin in the olefin feed introduced via line 103A. Additionally, only a small portion of the isoparaffin feed may be converted by the reaction in first stage 101A (such as about 10 wt % or less), 5 wt % or less, 2 wt % or less, 1 wt % or less, 0.5 wt % or less, or 0.1 wt % or less of the isoparaffin feed is converted based on the total weight of isoparaffin introduced to first stage 101A. Therefore, the amount of isoparaffin introduced to interstage 109A and, therefore, introduced to second stage 101B is similar to that introduced to first stage 101A and the olefin introduced to interstage space 109A via line 103B and from the effluent of first stage 101A serves to bring the olefin level back up to a desired i:o ratio. For example, if an i:o ratio of 100:1 is introduced to first stage 101A and there is an olefin conversion of 100%, then the i:o ratio in second stage 101B would be ˜99:1, if no additional isoparaffin was added. The combination of isoparaffin and olefin is then introduced to second stage 101B, where the olefin may be converted in second stage 101B, such as about 90 wt % or greater, 95 wt % or greater, 98 wt % or greater, or 99 wt % or greater is converted in second stage 101B, based on the total weight of olefin introduced to interstage space 109A. Similarly, only a small portion of the isoparaffin feed may be converted by the reaction in second stage 101B, such as about 10 wt % or less, 5 wt % or less, 2 wt % or less, 1 wt % or less, 0.5 wt % or less, or 0.1 wt % or less of the isoparaffin feed is converted based on the total weight of isoparaffin introduced to interstage space 109A. As the isoparaffin feed enters additional interstage spaces (such as second interstage space 109B, and third interstage space 109C) more olefin may be introduced (via lines 103C and 103D) to adjust the i:o ratio as the combined feeds are introduced to additional stages (such as third stage 101C and fourth stage 101D). Additionally, because the selected isoparaffin may be consumed in each stage, such as in amounts of about 10 wt % or less, about 5 wt % or less, about 2 wt % or less, about 1 wt % or less, about 0.5 wt % or less, or about 0.1 wt % or less, additional isoparaffin may be added in an interstage space so as to maintain a consistent i:o ratio throughout the multistage reactor. Similarly to FIG. 1A, an alkylation product mixture exits the reactor through line 107.

FIG. 1D depicts a multistage alkylation reactor 100D with eight reactor stages: first stage 101A, second stage 101B, third stage 101C, fourth stage 101D. The olefin feed is introduced to the reactor beds via lines 103A, 103B, 103C and 103D and OLHSV values are from about 0.01 hr⁻¹ to about 10 hr⁻¹, such as about 0.02 hr⁻¹ to about 1 hr⁻¹, or such as about 0.03 hr⁻¹ to about 0.1 hr⁻¹. The split introduction of the olefin feed allows a lower concentration (one quarter) of the olefin feed to be introduced locally to each of the first stage 101A, second stage 101B, third stage 101C, fourth stage 101D, fifth stage 101E, sixth stage 101F, seventh stage 101G, and eighth stage 101H. The isoparaffin feed is introduced to alkylation reactor 100D through line 105. Alkylation reactor 100D has multiple interstage spaces: first interstage space 109A, second interstage space 109B, third interstage space 109C, fourth interstage space 109D, fifth interstage space 109E, sixth interstage space 109F, and seventh interstage space 109G between reactor stages 101A, 101B, 101C, 101D, 101E, 101F, 101G, and 101H to allow for introduction of additional olefin feed through lines 103B, 103C, 103D, 103E, 103F, 103G, and 103H. If lines 103 and 105 have the same composition as in FIG. 1A, then the local i:o ratio is 8 times that found in FIG. 1A, because the olefin feed is divided into eight lines 103A, 103B, 103C, 103D, 103E, 103F, 103G, and 103H. The i:o ratio in a single stage is only slightly affected by prior stage(s) because the olefin introduced to a prior stage can be largely converted within that stage, but the isoparaffin is introduced at such a ratio that the amount converted may have little effect on the ratio in later stages. For example, the olefin introduced via line 103A to first stage 101A is converted, such as about 90 wt % or greater, about 95 wt % or greater, about 98 wt % or greater, or about 99 wt % or greater is converted in first stage 101A, based on the total weight of olefin in the olefin feed introduced via line 103A. Additionally, only a small portion of the isoparaffin feed may be converted by the reaction in first stage 101A, such as about 10 wt % or less of the isoparaffin feed is converted based on the total weight of isoparaffin introduced to first stage 101A, such as about 5 wt % or less, about 2 wt % or less, about 1 wt % or less, about 0.5 wt % or less, or about 0.1 wt % or less. Therefore, the amount of isoparaffin introduced to interstage 109A and, therefore, introduced to second stage 101B is similar or slightly less than that introduced to first stage 101A and the olefin introduced to interstage space 109A via line 103B and from the effluent of first stage 101A serves to bring the olefin level back up to a desired i:o ratio. Therefore, for example, if an i:o ratio of 100:1 is introduced to first stage 101A and there is an olefin conversion of 100% then the i:o ratio in second stage 101B would be ˜99:1, if no additional isoparaffin was added. The combination of isoparaffin and olefin is then introduced to second stage 101B, where the olefin may be converted in second stage 101B, such as about 90 wt % or greater, about 95 wt % or greater, about 98 wt % or greater, or about 99 wt % or greater is converted in second stage 101B, based on the total weight of olefin introduced to interstage space 109A. Similarly, only a small portion of the isoparaffin feed may be converted by the reaction in second stage 101B, such as about 10 wt % or less of the isoparaffin feed is converted based on the total weight of isoparaffin introduced to interstage space 109A, such as about 5 wt % or less, about 2 wt % or less, about 1 wt % or less, about 0.5 wt % or less, or about 0.1 wt % or less. As the isoparaffin feed enters additional interstage spaces (such as second interstage space 109B, and third interstage space 109C) more olefin may be introduced (via lines 103C, 103D, 103E, 103F, 103G, and 103H) to adjust the i:o ratio as the combined feeds are introduced to additional stages (such as third stage 101C, fourth interstage space 109D, fifth interstage space 109E, sixth interstage space 109F, and seventh interstage space 109G). Additionally, because the selected isoparaffin may be consumed in each stage, such as in amounts of about 10 wt % or less, about 5 wt % or less, about 2 wt % or less, about 1 wt % or less, about 0.5 wt % or less, or about 0.1 wt % or less, additional isoparaffin may be added in an interstage space so as to maintain a consistent i:o ratio throughout the multistage reactor. Similarly to FIG. 1A, an alkylation product mixture exits the reactor through line 107.

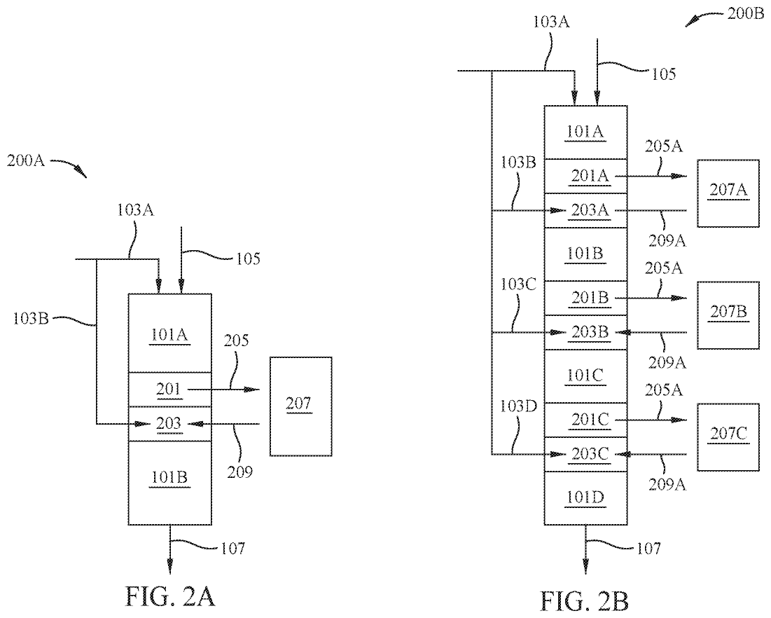

FIG. 2A depicts a multistage alkylation reactor 200A with two reactor stages: first stage 101A and second stage 101B. The olefin feed is introduced to the reactor beds via lines 103A and 103B and OLHSV values are from about 0.01 hr⁻¹ to about 10 hr⁻¹, such as about 0.02 hr⁻¹ to about 1 hr⁻¹, such as about 0.03 hr⁻¹ to about 0.1 hr⁻¹. The split introduction of the olefin feed allows a lower concentration (half) of the olefin feed to be introduced locally to each of the first stage 101A and the second stage 101B. The isoparaffin feed is introduced to alkylation reactor 200A through line 105. Alkylation reactor 200A has a divided interstage space including an outlet space 201 and an inlet space 203 between first stage 101A and second stage 101B to allow for removal and separation of the product mixture formed in first stage 101A (and introduction of oligomer light stream from separation system 207). As used herein, a “space” is a channel defined by proximate stage(s) or defined by a proximate stage and a proximate interstage barrier (e.g., a wall dividing adjacent spaces). The product of reactor stage 101A may be removed from outlet space 201 through line 205 to separation system 207 for removal of olefin oligomers and to form an oligomer light stream. Separation system 207 may be a distillation system, an adsorbent bed or other separations system capable of separating olefin oligomers from the remainder of the materials produced in reactor stage 101A. After separation, the oligomer light stream may be introduced to the multistage reactor in inlet space 203 via line 209. Furthermore, additional olefin feed may be introduced to inlet space 203 through line 103B.

FIG. 2B depicts a multistage alkylation reactor 200B with four reactor stages: first stage 101A, second stage 101B, third stage 101C, and fourth stage 101D. The olefin feed is introduced to the reactor beds via lines 103A, 103B, 103C and 103D and OLHSV values are from about 0.01 hr⁻¹ to about 10 hr⁻¹, such as about 0.02 hr⁻¹ to about 1 hr⁻¹, such as about 0.03 hr⁻¹ to about 0.1 hr⁻¹. The split introduction of the olefin feed allows a lower concentration (one quarter) of the olefin feed to be introduced locally to each of the first stage 101A, second stage 101B, third stage 101C, and fourth stage 101D. The isoparaffin feed is introduced to alkylation reactor 200B through line 105. Alkylation reactor 200B has multiple divided interstage spaces including first outlet space 201A, a second outlet space 201B, third outlet space 201C, first inlet space 203A, second inlet space 203B, and third inlet space 203C between reactor stages 101A, 101B, 101C, and 101D to allow for removal, separation, and reintroduction of the product mixture formed in first stage 101A, second interstage 101B, and third interstage 101C. The product of reactor stage 101A may be removed from outlet space 201A through line 205A to separation system 207A for removal of olefin oligomers and to form an oligomer light stream. After separation, the oligomer light stream may be introduced to the multistage reactor in inlet space 203A via line 209A. Furthermore, additional olefin feed may be introduced to inlet space 203A through line 103B. Separation system 207A, 207B, and 207C may be a distillation system, an adsorbent bed or other separations system capable of separating olefin oligomers from the remainder of the materials produced in reactor stage 101A.

Similar to FIG. 1C, first inlet space 203A, second inlet space 203B, and third inlet space 203C allow for introduction of additional olefin feed through lines 103B, 103C, and 103D. If lines 103 and 105 have a similar or the same composition as a composition of FIG. 1A, then the local i:o ratio is 4 times that found in FIG. 1A, because the olefin feed is divided into four lines 103A, 103B, 103C, and 103D. The i:o ratio in a single stage might only be slightly affected by prior stage(s) because the olefin introduced to a prior stage can be largely converted within that stage, but the isoparaffin is introduced at such a ratio that the amount converted may have little effect on the ratio in later stages. For example, the olefin introduced via line 103A to first stage 101A is converted, such as about 90 wt % or greater, 95 wt % or greater, 98 wt % or greater, or 99 wt % or greater is converted in first stage 101A, based on the total weight of olefin in the olefin feed introduced via line 103A. Additionally, only a small portion of the isoparaffin feed might be converted by the reaction in first stage 101A (such as about 10 wt % or less), 5 wt % or less, 2 wt % or less, 1 wt % or less, 0.5 wt % or less, 0.1 wt % or less, 0.05 wt % or less, or 0.01 wt % or less of the isoparaffin feed is converted based on the total weight of isoparaffin introduced to first stage 101A. Therefore, the amount of isoparaffin introduced to interstage outlet space 201A, and therefore introduced to inlet space 203A, and from there to second stage 101B, is similar to the amount of isoparaffin introduced to first stage 101A. The olefin introduced to inlet space 203A via line 103B and the olefin from the effluent of first separation system 207A can increase the olefin level to a desired i:o ratio. The combination of isoparaffin and olefin is then introduced to second stage 101B, where the olefin may be converted in second stage 101B, such as about 90 wt % or greater, 95 wt % or greater, 98 wt % or greater, or 99 wt % or greater of olefin is converted in second stage 101B, based on the total weight of olefin introduced to first inlet space 203A. Similarly, a small portion of the isoparaffin feed may be converted by the reaction in second stage 101B, such as about 10 wt % or less, 5 wt % or less, 2 wt % or less, 1 wt % or less, 0.5 wt % or less, 0.1 wt % or less, 0.05 wt % or less, or 0.01 wt % or less of the isoparaffin feed is converted based on the total weight of isoparaffin introduced to outlet space 201B. As the isoparaffin feed enters additional inlet spaces (such as second inlet space 203B, and third inlet space 203C) more olefin may be introduced (via lines 103C and 103D) to adjust the i:o ratio as the combined feeds are introduced to additional stages (such as third stage 101C and fourth stage 101D). Similarly to FIG. 1A, an alkylation product mixture exits the reactor through line 107.

### Separation System

Any suitable distillation apparatus may be used as a separation system to separate high boiling olefin oligomers from the product mixture. Distillation may be performed at a temperature of about 250° F. or greater, about 275° F. or greater, about 300° F. or greater, about 325° F. or greater, about 350° F. or greater, about 375° F. or greater, about 400° F. or greater, or about 450° F. or greater, such as from about 250° F. to about 600° F., from about 275° F. to about 550° F., from about 300° F. to about 500° F., or from about 350° F. to about 450° F. Furthermore, distillation may be performed at pressures greater or less than atmospheric pressure, such as pressures of about 1 kPa (about 0.145 psi) or greater, about 5 kPa (about 0.725 psi) or greater, about 10 kPa (about 1.45 psi) or greater, about 50 kPa (about 7.25 psi) or greater, about 100 kPa (about 14.5 psi) or greater, about 500 kPa (about 72.5 psi) or greater, about 1,000 kPa (about 145 psi) or greater, about 2,000 kPa (about 290 psi) or greater, about 3,000 kPa (about 435 psi) or greater, about 4,000 kPa (about 580 psi) or greater, about 5,000 kPa (about 725 psi) or greater, or about 5,500 kPa (about 798 psi) or greater, such as from about 1 kPa to about 12,000 kPa (about 0.145 psi to 1740 psi), about 100 kPa to about 8500 kPa (about 14.5 psi to 1233 psi), about 1,000 kPa to about 7,000 kPa (about 145 psi to 1015 psi), about 3000 kPa to about 7,000 kPa (about 435 psi to 1015 psi), about 4,500 kPa to about 7,000 kPa (about 653 psi to 1015 psi), about 5,000 kPa to about 6,500 kPa (about 725 psi to 943 psi), or about 5,500 kPa to about 6,500 kPa (about 798 to 943 psi). In at least one embodiment, the distillation pressure matches that of the multistage reactor. In some embodiments, the separation takes place at a pressure lower than the reactor pressure, where the decrease in pressure causes evaporation of reactants and product paraffin.

Furthermore, any suitable adsorption bed or process may be used to separate olefin oligomers from the product mixtures. For example, the adsorption bed may include a fixed or fluidized bed of solid or liquid adsorbent (e.g., ion exchange resin beds, activated carbon beds). The adsorption process may include processes that are diffusion limited or pressurized flow. The adsorbent may include materials such as clay, alumina, silica, titania, zirconia, aluminosilicates, zeolites, carbon, or combination(s) thereof. The adsorbent may be impregnated, coated, doped, or otherwise include metal atoms to aid in removal of olefin oligomers including transition metals, such as silver, platinum, palladium, ruthenium, nickel, iridium, iron, molybdenum, or cobalt.

The adsorbent may be regenerable, meaning that the ability to adsorb olefins may be regenerated. Regeneration of adsorbent materials may be performed by thermal, pressure, chemical, or other means. For example, in some embodiments, olefins may be desorbed from an adsorbent bed via reverse flow of inert gas at higher temperatures.

The adsorption process may take place at pressures greater or less than atmospheric pressure, such as pressures of about 1 kPa (about 0.145 psi) or greater, about 5 kPa (about 0.725 psi) or greater, about 10 kPa (about 1.45 psi) or greater, about 50 kPa (about 7.25 psi) or greater, about 100 kPa (about 14.5 psi) or greater, about 500 kPa (about 72.5 psi) or greater, about 1,000 kPa (about 145 psi) or greater, about 2,000 kPa (about 290 psi) or greater, about 3,000 kPa (about 435 psi) or greater, about 4,000 kPa (about 580 psi) or greater, about 5,000 kPa (about 725 psi) or greater, or about 5,500 kPa (about 798 psi) or greater, such as about 1 kPa to about 12,000 kPa (about 0.145 psi to 1740 psi), about 100 kPa to about 8500 kPa (about 14.5 psi to 1233 psi), about 1,000 kPa to about 7,000 kPa (about 145 psi to 1015 psi), about 3000 kPa to about 7,000 kPa (about 435 psi to 1015 psi), about 4,500 kPa to about 7,000 kPa (about 653 psi to 1015 psi), about 5,000 kPa to about 6,500 kPa (about 725 psi to 943 psi), or about 5,500 kPa to about 6,500 kPa (about 798 to 943 psi). In at least one embodiment, the adsorption pressure matches that of the multistage reactor. In some embodiments, adsorption is performed at a pressure higher than the reactor pressure, where the increase in pressure promotes condensation of gaseous reactants or products.

Adsorption may be performed at a temperature of about 50° F. or greater, about 100° F. or greater, about 150° F. or greater, about 200° F. or greater, about 250° F. or greater, or about 300° F. or greater, to about 350° F. or less, such as from about 50° F. to about 350° F., from about 100° F. to about 340° F., from about 150° F. to about 330° F., or from about 200° F. to about 320° F.

The separation system may remove all or a portion of the C5+ olefins in an alkylation product mixture. For example, the separation system may remove about 50 wt % or greater, about 60 wt % or greater, about 70 wt % or greater, about 80 wt % or greater, about 90 wt % or greater, about 95 wt % or greater, about 98 wt % or greater, about 99 wt % or greater, or substantially all of the C5+ olefins based on the weight of C5+ olefins in the alkylation product mixture.

### Feedstocks

Feedstocks useful in the present alkylation process include at least one isoparaffin feed and at least one olefin feed. The isoparaffin feed used in alkylation processes of the present disclosure may have from about 4 to about 7 carbon atoms. Representative examples of such isoparaffins include isobutane, isopentane, 3-methylhexane, 2-methylhexane, 2,3-dimethylbutane, and mixture(s) thereof, typically isobutane.

The olefin component of the feedstock may include at least one olefin having from 2 to 12 carbon atoms. Representative examples of such olefins include 2-butene, isobutylene, 1-butene, propylene, ethylene, pentene, hexene, octene, heptene, or mixture(s) thereof. In some embodiments, the olefin component of the feedstock is selected from the group consisting of propylene, butene, pentene and mixture(s) thereof. For example, in one embodiment, the olefin component of the feedstock may include a mixture of propylene and at least one butene, such as 2-butene, where the weight ratio of propylene to butene is from about 0.01:1 to about 1.5:1, such as from about 0.1:1 to about 1:1. In another embodiment, the olefin component of the feedstock may include a mixture of propylene and at least one pentene, where the weight ratio of propylene to pentene is from about 0.01:1 to about 1.5:1, such as from about 0.1:1 to about 1:1.

The concentration of olefin feed can be adjusted by, e.g., staged additions thereof. By staged additions, isoparaffin/olefin feed concentrations (and therefore the i:o ratio) can be maintained at levels to improve conversion and reduce catalyst deactivation. In at least one embodiment, the ratio of isoparaffin to olefin ratio by volume, referred to as the i:o ratio is: about 100:1 or greater, about 120:1 or greater, about 140:1 or greater, about 160:1 or greater, about 180:1 or greater, about 200:1 or greater, about 220:1 or greater, about 240:1 or greater, about 260:1 or greater, about 280:1 or greater, or about 300:1 or greater, such as from about 100:1 to about 500:1, about 120:1 to about 500:1, about 160:1 to about 480:1, about 200:1 to about 450:1, about 220:1 to about 450:1, about 240:1 to about 420:1, or about 240:1 to about 400:1.

The production of olefin oligomers increases with lower i:o ratios. To reduce or eliminate the production of olefin oligomers an i:o ratio of about 100:1 or greater may be used. On the other hand, the efficiency of the alkylation process can be reduced at higher i:o ratios, due to large quantity of isoparaffin present in the alkylation product mixture, which is then separated and recycled to the reactor. The separation and recycling of isoparaffin may occur in a distillation apparatus that allows for distillation of low C5− alkane from C6+ alkanes and alkenes produced in the reactor. A higher i:o ratio can provide greater quantities of C5− alkane separated from the alkylation product mixture that can be recycled to the reactor.

Before being sent to the reactor, the isoparaffin feed and/or olefin feed may be treated to remove catalyst poisons. For example, catalyst poisons may be removed using guard beds with specific absorbents for reducing the level of S, N, and/or oxygenates to values which do not affect catalyst stability, activity, and selectivity.

### Catalysts

One class of catalysts suitable for use in a process of this disclosure is a molecular sieve or zeolite. The molecular sieve may have a Constraint Index of about 5 or less, and may be a crystalline microporous material of the MWW framework type. MWW framework type refers to a type of crystalline microporous material that includes at least two independent sets of 10-membered ring channels and has composite building units of d6r (t-hpr) and mel as defined and discussed in Compendium of Zeolite Framework Types. Building Schemes and Type Characteristics Van Koningsveld, Henk, (Elsevier, Amsterdam, 2007), incorporated by reference. Crystalline microporous materials of the MWW framework type can include those molecular sieves having an X-ray diffraction pattern including d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system. Crystalline microporous materials of the MWW framework type include molecular sieves having natural tiling units of t-dac-1, t-euo, t-hpr, t-kah, t-kzd, t-mel, t-mww-1, t-mww-2, and t-srs as defined and discussed in Three-periodic Nets and Tilings: Natural Tilings for Nets, V. A. Blatov, O. Delgado-Friedrichs, M. O'Keeffe and D. M. Proserpio, Acta Crystallogr. A 63, 418-425 (2007), incorporated by reference.

In at least one embodiment, the crystalline microporous material is a zeolite. As used herein, the term “crystalline microporous material of the MWW framework type” includes one or more of:

(a) molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, incorporated herein by reference);

(b) molecular sieves made from a second degree building block, being a 2-dimensional tiling of such MWW framework topology unit cells, forming a monolayer of one unit cell thickness, in one embodiment, one c-unit cell thickness;

(c) molecular sieves made from common second degree building blocks, being layers of one or more than one unit cell thickness, where the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof and

(d) molecular sieves made by any regular or random 2-dimensional or 3-dimensional combination of unit cells having the MWW framework topology.

Examples of crystalline microporous materials of the MWW framework type include MCM-22 (U.S. Pat. No. 4,954,325), PSH-3 (U.S. Pat. No. 4,439,409), SSZ-25 (U.S. Pat. No. 4,826,667), ERB-1 (European Patent No. 0293032), ITQ-1 (U.S. Pat. No. 6,077,498), ITQ-2 (International Publication No. WO97/17290), MCM-36 (U.S. Pat. No. 5,250,277), MCM-49 (U.S. Pat. No. 5,236,575), MCM-56 (U.S. Pat. No. 5,362,697), UZM-8 (U.S. Pat. No. 6,756,030), UZM-8HS (U.S. Pat. No. 7,713,513), UZM-37 (U.S. Pat. No. 7,982,084), EMM-10 (U.S. Pat. No. 7,842,277), EMM-12 (U.S. Pat. No. 8,704,025), EMM-13 (U.S. Pat. No. 8,704,023), UCB-3 (U.S. Pat. No. 9,790,143B2), MIT-1 (Luo, et. al., Chem Sci. 2015 Nov. 1; 6(11): 6320-6324), and mixtures thereof.

In some embodiments, the crystalline microporous material of the MWW framework type may be contaminated with other crystalline materials, such as ferrierite or quartz. These contaminants may be present in quantities of about 10 wt % or less, such as about 5 wt % or less. In some embodiments, the crystalline microporous material of the MWW framework type employed may be an aluminosilicate material having a silica to alumina molar ratio of about 10 or more, such as from about 10 to about 50.

### Binder

Catalysts suitable for use in the systems and processes described include a binder.

Binder materials, may include inorganic oxides, such as alumina, silica, titanic, zirconia and mixtures and compounds thereof, may be present in the catalyst in amounts about 90 wt % or less, for example about 80 wt % or less, such as about 70 wt % or less, for example about 60 wt % or less, such as about 50 wt % or less. Where a non-alumina binder is present, the amount employed may be about 1 wt % or more, such as about 5 wt % or more, for example about 10 wt % or more. In at least one embodiment, a silica binder is employed such as disclosed in U.S. Pat. No. 5,053,374, incorporated by reference. In other embodiments, a zirconia or titania binder is used.

In other embodiments, the binder may be a crystalline oxide material such as the zeolite-bound-zeolites described in U.S. Pat. Nos. 5,665,325 and 5,993,642, incorporated by reference. In the case of crystalline binders, the binder material may contain alumina, including amorphous alumina.

### Product

The product of the alkylation reaction (also referred to as the alkylation product mixture) can include: alkanes resulting from the alkylation of isoparaffin with olefin, unreacted isoparaffin, unreacted olefin, olefin oligomers, other byproducts, including other alkanes and alkenes. The product composition of the isoparaffin-olefin alkylation reaction described is dependent on the reaction conditions and the composition of the olefin feed and isoparaffin feed. The product is a complex mixture of hydrocarbons, since alkylation of the feed isoparaffin by the feed olefin is accompanied by a variety of competing reactions including cracking, olefin oligomerization, and/or further alkylation of the alkylate product by the feed olefin. For example, in the case of alkylation of isobutane with C3-C5 olefins, such as 2-butene, the product may include about 20-30 wt % of C5-C7 hydrocarbons, 50-75 wt % of C8 hydrocarbons and 2.5-20 wt % of C9+ hydrocarbons. Moreover, using an MWW type molecular sieve as the catalyst, it has been discovered that processes can be selective to desirable high octane components so that, in the case of alkylation of isobutane with C3-C5 olefins, the C6 fraction typically includes at least 40 wt %, such as at least 70 wt %, of 2,3-dimethylbutane, the C7 fraction typically includes at least 40 wt %, such as at least 80 wt %, of 2,3-dimethylpentane and the C8 fraction typically includes at least 50 wt %, such as at least 70 wt %, of 2,3,4-trimethylpentane, 2,3,3-trimethylpentane, and 2,2,4-trimethylpentane.

Additionally, in the case of alkylation of isobutane with C5 olefins, such as n-pentene and 2-methyl-2-butene, the product may include about 30-40 wt % of C5 hydrocarbons, 15-25 wt % of C9 hydrocarbons, 25-35 wt % of C8 hydrocarbons, and 2.5-10 wt % of C10+ hydrocarbons. Moreover, using an MWW type molecular sieve as the catalyst, it has been found that a process can be selective to desirable high octane components so that, in the case of alkylation of isobutane with C5 olefins, the C8 and C9 fractions typically include a higher molar ratio of trimethyl isomers to dimethyl isomers, which is beneficial for increasing octane. For the C8 fraction, the molar ratio of trimethylpentane to dimethylhexane can be about 3 or more, e.g. about 4 to about 5, or about 3 to about 6. For the C9 fraction, the molar ratio of trimethylhexane to dimethylheptane can be about 1 or more, e.g. about 1.5 or more, or from about 1 to about 3.

The product of the isoparaffin-olefin alkylation reaction may be fed to a separation system, such as a distillation train, to recover a C5+ fraction for use as a gasoline octane enhancer. Additionally, the separation system may separate the C4-C6 isoparaffin to be recycled as part or all of the isoparaffin feed. Furthermore, depending on alkylate demand, part or all of a C9+ fraction can be recovered for use as a distillate blending stock.

### Other Embodiments of the Present Disclosure

Clause 1. A process for the alkylation of an isoparaffin, the process including:

introducing, in a multistage reactor, a solid acid catalyst to an isoparaffin feed and an olefin feed to form an alkylation product mixture;

separating at least a portion of C5+ olefins from the alkylation product mixture to form an oligomer light stream; and

where the solid acid catalyst includes a zeolite.

Clause 2. The process of clause 1, where the zeolite is a crystalline microporous material of the MWW framework type.

Clause 3. The process of clause 2, where the crystalline microporous material of the MWW framework types is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, UCB-3, or mixture(s) thereof.

Clause 4. The process of clause 3, where the crystalline microporous material of the MWW framework types is selected from the group consisting of MCM-22, MCM-49, MCM-56, EMM-10, or mixture(s) thereof.

Clause 5. The process of any of clauses 1 to 4, where the oligomer light stream is introduced to the multistage reactor.

Clause 6. The process of any of clauses 1 to 5, where the multistage reactor includes a first stage and a second stage.

Clause 7. The process of clause 6, where the oligomer light stream is introduced to the multistage reactor downstream of the first stage.

Clause 8. The process of any of clauses 1 to 7, where separating is performed by distillation.

Clause 9. The process of any of clauses 1 to 7, where the separating is performed by adsorption.

Clause 10. The process of any of clauses 1 to 9, where the at least a portion of C5+ olefins separated is greater than 50 wt %.

Clause 11. The process of any of clauses 1 to 10, where the oligomer light stream is substantially free of C5+ olefins.

Clause 12. The process of any of clauses 1 to 11, where the isoparaffin feed and the olefin feed are introduced to the multistage reactor at a ratio of isoparaffin feed to olefin feed of about 100:1 or greater.

Clause 13. A multistage reactor for the alkylation of an isoparaffin with an olefin, the multistage reactor including:

a first stage;

a second stage;

a separation system;

a first outlet space coupling the first stage and the separation system;

a first inlet space coupling the separation system and the second stage;

a first inlet disposed upstream of the first stage and configured to receive an olefin feed; and

a second inlet disposed upstream of the first stage and configured to receive an isoparaffin feed.

Clause 14. The multistage reactor of clause 13, further including a first outlet disposed downstream of the first stage coupled with the separation system and configured to release a first alkylation product mixture.

Clause 15. The multistage reactor of any of clauses 13 to 14, further including a third inlet disposed downstream of the first stage coupled with the separation system and configured to receive an oligomer light stream.

Clause 16. The multistage reactor of any of clauses 13 to 15, further including an outlet configured to release an alkylation product mixture.

Clause 17. The multistage reactor of any of clauses 13 to 16, where the separation system is a distillation apparatus.

Clause 18. The multistage reactor of any of clauses 13 to 16, where the separation system includes one or more adsorbent beds.

Clause 19. The multistage reactor of clause 18, where the one or more adsorbent beds include a molecular sieve.

Clause 20. The multistage reactor of clause 18, where the one or more adsorbent beds include carbon.

Clause 21. A multistage reactor for the alkylation of an isoparaffin with an olefin, the multistage reactor including:

a first stage;

a first inlet disposed upstream of the first stage and configured to receive an olefin feed;

a second inlet disposed upstream of the first stage and configured to receive an isoparaffin feed;

a first separation system;

a second stage;

a first outlet space coupling the first stage and the first separation system;

a first inlet space coupling the first separation system and the second stage;

a third stage;

a second separation system;

a second outlet space coupling the second stage and the second separation system;

a second inlet space coupling the second separation system and the third stage; and

an outlet configured to release an alkylation product mixture.

Clause 22. The multistage reactor of clause 21, where the first separation system and the second separation system are independently a distillation apparatus or one or more adsorption beds.

Clause 23. The multistage reactor of any of clauses 20 to 21, further including:

a fourth stage;

a fifth stage;

a sixth stage;

a seventh stage; and

an eighth stage.

Clause 24. The multistage reactor of clause 23, further including:

a third separation system;

a third outlet space coupling the third stage and the third separation system;

a third inlet space coupling the third separation system and the fourth stage;

a fourth separation system;

a fourth outlet space coupling the fourth stage and the fourth separation system;

a fourth inlet space coupling the fourth separation system and the fifth stage;

a fifth separation system;

a fifth outlet space coupling the fifth stage and the fifth separation system;

a fifth inlet space coupling the fifth separation system and the sixth stage;

a sixth separation system;

a sixth outlet space coupling the sixth stage and the sixth separation system;

a sixth inlet space coupling the sixth separation system and the seventh stage;

a seventh separation system;

a seventh outlet space coupling the seventh stage and the seventh separation system; and

a seventh inlet space coupling the seventh separation system and the eighth stage.

Clause 25. The multistage reactor of clause 24, further including:

a ninth stage; and

a tenth stage.

Clause 26. The multistage reactor of clause 25, further including:

an eighth separation system;

an eighth outlet space coupling the eighth stage and the eighth separation system;

an eighth inlet space coupling the eighth separation system and the ninth stage;

a ninth separation system;

a ninth outlet space coupling the ninth stage and the ninth separation system;

a ninth inlet space coupling the ninth separation system and the tenth stage;

Clause 25. The multistage reactor of clause 24, further comprising:

an eleventh stage; and

a twelfth stage.

Clause 26. The multistage reactor of clause 25, further including:

a tenth separation system;

a tenth outlet space coupling the tenth stage and the tenth separation system;

a tenth inlet space coupling the tenth separation system and the eleventh stage;

an eleventh separation system;

an eleventh outlet space coupling the eleventh stage and the eleventh separation system; and

an eleventh inlet space coupling the eleventh separation system and the twelfth stage.

### EXAMPLES

### Feed Pretreatment

Isobutane was obtained from a commercial source and used as received. The isobutene purity was 99.6% with the balance n-butane.

Propylene and 2-butene were obtained from a commercial specialty gases source and used as received. The 2-butene was a mixture of trans-2-butene and cis-2-butene.

### Catalyst Preparation and Loading

Catalysts used for isobutene alkylation with light olefins are dried in the reactor under nitrogen flow at 250° C. for at least 4 hours prior to use.

### Example 1

The catalyst was prepared by combining 80 parts MCM-49 zeolite crystals with 20 parts pseudoboehmite alumina, on a calcined dry weight basis. The MCM-49 and pseudoboehmite alumina dry powder were placed in a muller or a mixer and mixed for 30 minutes. Sufficient water was added to the MCM-49 and alumina during the mixing process to produce an extrudable paste. The extrudable paste was formed into a 1/20 inch quadralobe extrudate using an extruder. After extrusion, the extrudate was dried at a temperature ranging from 250° F. (121° C.) to 325° F. (168° C.). After drying, the dried extrudate was heated to 1000° F. (538° C.) under flowing nitrogen. The extrudate was then cooled to ambient temperature, humidified with saturated air or steam and then ion exchanged with 0.75 N ammonium nitrate solution followed by washing with deionized water and drying. The extrudate was then calcined in a nitrogen/air mixture to a temperature of 1000° F. (538° C.).

### Example 2

95 parts MCM-49 zeolite crystals were combined with 5 parts pseudoboehmite alumina, on a calcined dry weight basis. The MCM-49 and pseudoboehmite alumina dry powder were placed in a muller or a mixer and mixed for 30 minutes. Sufficient water was added to the MCM-49 and alumina during the mixing process to produce an extrudable paste. The extrudable paste was formed into a 1/20 inch quadralobe extrudate using an extruder. After extrusion, the extrudate was dried at a temperature ranging from 250° F. (121° C.) to 325° F. (168° C.). After drying, the dried extrudate was heated to 1000° F. (538° C.) under flowing nitrogen. The extrudate was then cooled to ambient temperature, humidified with saturated air or steam and then ion exchanged with 0.75 N ammonium nitrate solution followed by washing with deionized water and drying. The extrudate was then calcined in a nitrogen/air mixture to a temperature of 1000° F. (538° C.).

### Example 3

The catalyst of Example 1 was loaded into a pilot plant and operated as a single stage reactor, as shown in FIG. 1A. The reactor was 60″ long and made from ¾″ O.D. Schedule 40 pipe. The reactor was loaded with 50 g of catalyst. The reactor was located in an isothermal sand bath maintained at 302° F. (150° C.). Reactor pressure was 750 psig (5171 kPag). Isobutane (99.6% purity) and 2-butene were independently fed to the top of the single stage reactor at a relative rate such that the isobutane to 2-butene ratio at the top of the catalyst bed was 40:1. The reactor effluent was measured using a FID GC equipped with a 150 m Petrocol column. The 2-butene flow to the reactor was set to achieve an Olefin Liquid Hourly Space Velocity (OLHSV) of 0.06 h⁻¹ and subsequently 0.03 h⁻¹. Isobutane flowrates were adjusted as olefin flowrates were adjusted to maintain a constant i:o of 40:1 to the inlet to the catalyst bed. Average 2-butene conversion at 0.06 h⁻¹ was 80.7% and at 0.03 h⁻¹ the average 2-butene conversion was 93.7%.

### Example 4

The catalyst of Example 1 was loaded into a pilot plant and operated as a 2 stage reactor, as shown in FIG. 1B. Each stage was 60″ long and made from ¾″ O.D. Schedule 40 pipe. Each stage was loaded with 50 g of catalyst. The two-stage reactor was located in an isothermal sand bath maintained at 302° F. (150° C.). Reactor pressure was 750 psig (5171 kPag). Isobutane (99.6% purity) was fed to the first stage of the reactor and the 2-butene flow was split evenly into 2 using Coriolis meters and independently fed to each stage. The relative rates of isobutane and 2-butene were set such that the isobutane to 2-butene ratio at the top of the first stage was 40:1. The alkylation product mixture exiting the reactor was measured using a FID GC equipped with a 150 m Petrocol column. The total 2-butene flow to the reactor was set to achieve an Olefin Liquid Hourly Space Velocity (OLHSV) of 0.06 h⁻¹. Average 2-butene conversion at 0.06 h⁻¹ was 70.4%. As can be seen when comparing the results to Example 5, the operation of the 2 stage system resulted in significantly lower olefin conversion.

### Example 5

The catalyst of Example 1 was loaded into a pilot plant and operated as a 4-stage reactor, as shown in FIG. 1C. Each stage was 60″ long and made from ¾″ O.D. Schedule 40 pipe. Each stage was loaded with 50 g of catalyst. The four-stage reactor was located in an isothermal sand bath maintained at 302° F. (150° C.). Reactor pressure was 750 psig (5171 kPag). Isobutane (99.6% purity) was fed to the first stage and the 2-butene flow was split evenly into 4 using Coriolis meters and independently fed to each stage of the four-stage reactor. The relative rates of isobutane and 2-butene were set such that the isobutane to 2-butene ratio at the top of the first stage was 40:1. The alkylation product mixture exiting the reactor was measured using a FID GC equipped with a 150 m Petrocol column. The total 2-butene flow to the reactor was set to achieve an Olefin Liquid Hourly Space Velocity (OLHSV) of 0.06 h⁻¹ and subsequently 0.03 h⁻¹. Isobutane flowrates were adjusted as olefin flowrates were adjusted to maintain a constant i:o of 40:1 to the inlet to the first stage. Average 2-butene conversion at 0.06 h⁻¹ was 61.4% and at 0.03 h⁻¹ the average 2-butene conversion was 77.5%. As can be seen when comparing the results to Example 5, the operation of the 4 bed system resulted in significantly lower olefin conversion.

### Example 6

The catalyst of Example 2 was loaded into a pilot plant and operated as a 4 reactor bed system, as shown in FIG. 1(c). Each reactor was 60″ long and made from ¾″ O.D. Schedule 40 pipe. Each reactor was loaded with 150 g of catalyst. The reactors were located in an isothermal sandbath maintained at 302° F. (150° C.). Reactor pressure was 750 psig (5171 kPag). Isobutane (99.6% purity) was fed to the first reactor bed and the 2-butene flow was split evenly into 4 using Coriolis meters and independently fed to each reactor bed. The relative rates of isobutane and 2-butene were set such that the isobutane to 2-butene ratio at the top of the first reactor bed was ˜40:1. The reactor effluent exiting Bed 4 was measured using a FID GC equipped with a 150 m Petrocol column. The total 2-butene flow to the reactor was set to achieve an Olefin Liquid Hourly Space Velocity (OLHSV) of 0.018 h⁻¹. Average 2-butene conversion at ˜12 days on stream was ˜95%. The reactor effluent exiting Bed 4 was sent to a distillation column for separation of all C4 and lighter components from the reaction product. The C5+ alkylate was analyzed for olefinic content using ASTM D1159 and the resulting Bromine number was 10.6. Olefin content was estimated based on GC analysis to be ˜2.8 wt %.

### Example 7

The C5+ alkylate of Example 6 was submitted for distillation using a still with a separations capacity equivalent to 15 theoretical plates. The first target cut point was 300° F. The first cut, the <300° F. boiling material which included 81.4% of the weight of the original charge, had a Bromine number of 5.5, measured using ASTM D1159. The >300° F. boiling material, including 18.6% of the original charge, had a Bromine number of 31.8, measured using ASTM D1159. The >300° F. boiling material was then further distilled with a second target cut point of 400° F. The 300-400° F. boiling range material, including 88.8% of the second charge, had a Bromine number of 6.9 measured using ASTM D1159. The residual 400° F. boiling range material had a Bromine number of 57.9. As shown by this example, the heavy olefins produced by the alkylation reaction of Example 5 are concentrated in the high boiling range of the alkylate produced.

### Example 8

The catalyst of Example 2 was loaded into a pilot plant and operated as a 4-stage reactor, as shown in FIG. 1C. Each stage was 60″ long and made from ¾″ O.D. Schedule 40 pipe. Each stage was loaded with 150 g of catalyst. The reactor was located in an isothermal sand bath maintained at 302° F. (150° C.). Reactor pressure was 750 psig (5171 kPag). Isobutane (99.6% purity) was fed to the first stage and the 2-butene flow was split evenly into 4 using Coriolis meters and independently fed to each reactor bed. The relative rates of isobutane and 2-butene were set such that the isobutane to 2-butene ratio at the top of the first stage was 40:1. The alkylation product mixture exiting the multistage reactor was measured using a FID GC equipped to with a 150 m Petrocol column. The total 2-butene flow to the reactor was set to achieve an Olefin Liquid Hourly Space Velocity (OLHSV) of 0.018 h⁻¹. Average 2-butene conversion at ˜32 days on stream was ˜99.6%. The reactor effluent exiting Bed 4 was sent to a distillation column for separation of all C4 and lighter components from the reaction product. The alkylate produced was analyzed via offline GC and shown to have ˜2.3 wt % C5+ olefins and a Bromine number of 7.6 as measured by ASTM D1159.

### Example 9

The catalyst of Example 2 was loaded into a pilot plant and operated as a single bed, as shown in FIG. 1(a). The reactor was 14″ long and made from ⅜″ O.D. stainless steel tubing. The reactor was loaded with 4 g of catalyst. The reactor was located in an electrically heated furnace and maintained at 302° F. (150° C.). Reactor pressure was 750 psig (5171 kPag). A pre-mixed gas blend with isobutane and 2-butene at a 40:1 ratio was fed to the top of the reactor bed. The reactor effluent was measured using a FID GC equipped with a 150 m Petrocol column. The flow to the reactor was set to achieve an Olefin Liquid Hourly Space Velocity (OLHSV) of 0.057 h⁻¹. Average 2-butene conversion at 0.057 h⁻¹ was 99.7%.

### Example 10

The catalyst of Example 2 was loaded into a pilot plant single stage reactor, as shown in FIG. 1A. The reactor was 14″ long and made from ⅜″ O.D. stainless steel tubing. The reactor was loaded with 4 g of catalyst. The reactor was located in an electrically heated furnace and maintained at 302° F. (150° C.). Reactor pressure was 750 psig (5171 kPag). A pre-mixed gas blend with isobutane and 2-butene at a 40:1 ratio was fed to the top of the reactor. The alkylation product mixture was analyzed using a FID GC equipped with a 150 m Petrocol column. The flow to the reactor was set to achieve an Olefin Liquid Hourly Space Velocity (OLHSV) of 0.057 h⁻¹. To simulate the operation of bed 5 in a multistage reactor configuration, alkylate produced in a 4 stage unit in Example 8 was co-fed at a rate of 3.25 cc/hr. Average 2-butene conversion at 0.057 h⁻¹ was 58.1% at 10 days of cofeeding and continued to drop with days on stream. As demonstrated by this example, the presence of ˜2% C5+ olefins in the feed caused the 2-butene conversion to decrease by ˜41.6%.

### Example 11

A sample of the 4 stage alkylate produced in Example 8 was distilled to remove the components boiling at >300° F. The <300° F. alkylate was analyzed by GC and shown to have ˜3.4% C5+ olefins. The Bromine number of the <300° F. alkylate was 6.1. The catalyst of Example 2 was loaded into a pilot plant and operated as a single bed, as shown in FIG. 1(a). The reactor was 14″ long and made from ⅜″ O.D. stainless steel tubing. The reactor was loaded with 4 g of catalyst. The reactor was located in an electrically heated furnace and maintained at 302° F. (150° C.). Reactor pressure was 750 psig (5171 kPag). A pre-mixed gas blend with isobutane and 2-butene at a 40:1 ratio was fed to the top of the reactor bed. The reactor effluent was measured using a FID GC equipped with a 150 m Petrocol column. The flow to the reactor was set to achieve an Olefin Liquid Hourly Space Velocity (OLHSV) of 0.057 h⁻¹. To simulate the operation of bed 5 in a multistage reactor configuration where the heavier olefin content has been reduced, the <300° F. alkylate distilled above was co-fed at a rate of 3.25 cc/hr. Average 2-butene conversion at 0.057 h⁻¹ was 96.1%. As demonstrated by this example, the presence of ˜0.4% C5+ olefins in the feed caused the 2-butene conversion to decrease by ˜3.6% vs. the reference case in Example 9.

### Example 12

The catalyst of Example 2 was loaded into a pilot plant and operated as a 4 reactor bed system, as shown in FIG. 1(c). Each reactor was 60″ long and made from ¾″ O.D. Schedule 40 pipe. Each reactor was loaded with 150 g of catalyst. The reactors were located in an isothermal sandbath maintained at 266° F. (130° C.). Reactor pressure was 750 psig (5171 kPag). Isobutane (99.6% purity) was fed to the first reactor bed and the 2-butene flow was split evenly into 4 using Coriolis meters and independently fed to each reactor bed. The relative rates of isobutane and 2-butene were set such that the isobutane to 2-butene ratio at the top of the first reactor bed was ˜40:1. The reactor effluent exiting Bed 4 was measured using a FID GC equipped with a 150 m Petrocol column. The total 2-butene flow to the reactor was set to achieve an Olefin Liquid Hourly Space Velocity (OLHSV) of 0.018 h⁻¹. Average 2-butene conversion at ˜6 days on stream was ˜93%. The reactor effluent exiting Bed 4 was sent to a distillation column for separation of all C4 and lighter components from the reaction product. The alkylate produced was analyzed via offline GC and shown to have ˜7.1 wt % C5+ olefins and a Bromine number of 25.4 as measured by ASTM D1159.

### Example 13

The adsorption of olefins from the alkylate of Example 12 was illustrated as follows. Samples of solid adsorbents obtained from commercial vendors were dried overnight at 120° C. to remove adsorbed water, then allowed to cool to room temperature. One gram of the dried adsorbent was added to 10 g of the alkylate of Example 12 in a bottle and held for 2 hours with occasional shaking. After 2 hours, the solids were removed by filtration and the filtrate analyzed for composition by GC. Results for olefins are shown in Table 1 below and results for paraffins are shown in Table 2 below:

TABLE 1

| | Starting | G250 | Axsorb 913 | Molsieve | Molsieve | Activated |
|---|---|---|---|---|---|---|
| Olefin | Concentration | Alumina | (Axens) | 5Å | 13X | Carbon |
| 2-methyl-1- | 0.0107 | 8% | 9% | 13% | 12% | 21% |
| Trans-2-pentene | 0.038 | 5% | 5% | 15% | 10% | 25% |
| 4-methyl-trans-2- | 0.0188 | 79% | 93% | 62% | No Data | 72% |
| 3-methyl-cis-2- | 0.0056 | 0% | 5% | 0% | 7% | 20% |
| 3,4,4-trimethyl- | 0.0793 | 1% | 12% | 8% | 6% | 7% |

TABLE 2

| | Starting | G250 | Axsorb 913 | Molsieve | Molsieve | Activated |
|---|---|---|---|---|---|---|
| Paraffin | Concentration | Alumina | (Axens) | 5Å | 13X | Carbon |
| 2,3,- | 1.313 | 0% | 0% | 0% | 0% | 0% |
| 3-methylhexane | 0.239 | 0% | 0% | 0% | −1% | 0% |
| 2,2,4- | 1.929 | 0% | 0% | 0% | −1% | −1% |

As the data in the tables above show, adsorbents can be used to remove C5+ olefins from the alkylation product mixture without impacting the paraffins. As illustrated by the examples of this disclosure, the operation of a multi-stage isoparaffin alkylation reactor with interstage olefin injection is impacted by the production of heavier olefins. As heavier olefins are produced in early catalyst beds, they negatively impact the conversion in later catalyst beds, likely due to adsorption of the heavy olefins on the catalyst surface. Removal of these heavy olefins from the effluent of a catalyst bed prior to the introduction of the product mixture to the subsequent bed will minimize the impact on conversion. This removal can be done via physical separation schemes, such as distillation can be used to remove the highest boiling fraction of the alkylate where the heavy olefins are concentrated. Other separation schemes, for example membrane separation or adsorption could be deployed to affect the olefin removal from the alkylation product mixture. Overall, it has been discovered that certain byproducts, including olefin oligomers, produced during the alkylation of isoparaffins with olefins in a multistage reactor may decrease catalyst activity and reduce conversion of olefins. It has also been discovered that the produced olefin oligomers may be reduced or eliminated by interstage separation, which may include distillation, adsorption, or other separation methods. A multistage reactor may provide greater conversion and production rates and decrease overall costs of production. The combination of using a reactor having two or more stages and interstage separation of higher olefins may provide reduced olefin oligomers, increased olefin conversion, increased production, decreased catalyst deactivation, and/or improved product selectivity, as compared to multistage alkylation processes in the absence of olefin oligomer removal.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of this disclosure, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and/or testing procedures to the extent they are not inconsistent with this text. As is apparent from the foregoing general description and the specific embodiments, while forms of this disclosure have been illustrated and described, various modifications can be made without departing from the spirit and scope of this disclosure. Accordingly, it is not intended that this disclosure be limited thereby. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

While the present disclosure has been described with respect to a number of embodiments and examples, those skilled in the art, having benefit of this disclosure, will appreciate that other embodiments can be devised which do not depart from the scope and spirit of the present disclosure.

The invention claimed is:

1. A process for the alkylation of an isoparaffin, the process comprising:
introducing, in a multistage reactor, a solid acid catalyst comprising a zeolite to an isoparaffin feed and an olefin feed to form an alkylation product mixture comprising C5+ olefins; and
separating at least a portion of the C5+ olefins from the alkylation product mixture to form an oligomer light stream.

2. The process of claim 1, wherein the zeolite is a crystalline microporous material of the MWW framework type.

3. The process of claim 1, wherein the crystalline microporous materials of the MWW framework type is selected from the group consisting of MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, UCB-3, or mixture(s) thereof.

4. The process of claim 1, wherein the multistage reactor comprises a first stage and a second stage.

5. The process of claim 4, wherein the oligomer light stream is introduced to the multistage reactor downstream of the first stage.

6. The process of claim 1, wherein separating is performed by distillation.

7. The process of claim 1, wherein the separating is performed by adsorption.

8. The process of claim 1, wherein separating comprises separating greater than 50 wt % of at the C5+ olefins from the alkylation product mixture.

9. The process of claim 8, wherein the oligomer light stream is substantially free of C5+ olefins.

10. The process of claim 1, wherein the isoparaffin feed and the olefin feed are introduced to the multistage reactor at a ratio of isoparaffin feed to olefin feed of about 100:1 or greater.

11. A multistage reactor for the alkylation of an isoparaffin with an olefin, the multistage reactor comprising:
a first stage;
a second stage;
a separation system;
a first outlet space coupling the first stage and the separation system;
a first inlet space coupling the separation system and the second stage;
a first inlet disposed upstream of the first stage and configured to receive an olefin feed; and
a second inlet disposed upstream of the first stage and configured to receive an isoparaffin feed.

12. The multistage reactor of claim 11, further comprising a first outlet disposed downstream of the first stage coupled with the separation system and configured to release a first alkylation product mixture.

13. The multistage reactor of claim 11, further comprising a third inlet disposed downstream of the first stage coupled with the separation system and configured to receive an oligomer light stream.

14. The multistage reactor of claim 11, further comprising an outlet configured to release an alkylation product mixture.

15. The multistage reactor of claim 11, wherein the separation system is a distillation apparatus.

16. The multistage reactor of claim 11, wherein the separation system comprises one or more adsorbent beds.

17. The multistage reactor of claim 16, wherein the one or more adsorbent beds comprises a molecular sieve.

18. The multistage reactor of claim 16, wherein the one or more adsorbent beds comprises carbon.

19. A multistage reactor for the alkylation of an isoparaffin with an olefin, the multistage reactor comprising:
a first stage;
a first inlet disposed upstream of the first stage and configured to receive an olefin feed;
a second inlet disposed upstream of the first stage and configured to receive an isoparaffin feed;
a first separation system;
a second stage;
a first outlet space coupling the first stage and the first separation system;
a first inlet space coupling the first separation system and the second stage;
a third stage;
a second separation system;
a second outlet space coupling the second stage and the second separation system;
a second inlet space coupling the second separation system and the third stage; and
an outlet configured to release an alkylation product mixture.

20. The multistage reactor of claim 19, wherein the first separation system and the second separation system are independently a distillation apparatus or an adsorption bed.
